(19) United States
(12) Patent Application Publication
(10) Pub. No.: US 2005/0271613 A1
(43) Pub. Date: Dec. 8, 2005

(54) Beta-1, 3-1, 6-D-glucan and its use

(76) Inventors: Toshio Suzuki, Osaka-shi (JP); Seiji Nakamura, Osaka-shi (JP); Satoshi Nakayama, Osaka-shi (JP); Kouji Nishikawa, Osaka-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/090,052

(22) Filed: Mar. 28, 2005

(30) Foreign Application Priority Data
Mar. 29, 2004 (JP) 2004-095561
Sep. 9, 2004 (JP) 2004-262515

(51) Int. Cl. A61K 7/06; A61K 31/715; A61K 7/11

(52) U.S. Cl. 424/70.13; 514/54; 424/439

(57) ABSTRACT

An aqueous β-1,3-1,6-D-glucan solution, wherein ¹HNMR spectra on its solution containing in 1N aqueous sodium hydroxide heavy solution have two signals of 4.7 ppm and 4.5 ppm and the viscosity (BM type rotary-viscometer, 12 rpm) of its solution at 30° C., pH5.0, and its concentration 0.5% (w/v) is 50 cP ([mPa.s]) or less.

### TECHNICAL FIELD

The present invention relates to a polysaccharide, namely β-1,3-1,6-D-glucan useful for soft drinks, materials for healthy foods, thickeners for food and medicines obtainable from the culture medium containing β-glucan, especially β-1,3-1,6-D-glucan as a main ingredient, which a micoorganism, especially a microorganism belonging to Aureobasidium sp. produces extracellularly.

### BACKGROUND ART

It has become that β-glucan (β-1,3-D-glucan or β-1,6-D-glucan, or β-1,3-1,6-D-glucan) is an ingredient which is much contained in Lentinus edodes (Fruiting body of Basidiomycota) which naturally grow, and its culture mycelium. Furthermore, β-glucan is known to have immunomodulatory activity and anticancer activity. For example, β-glucan extracted from Shyzophyllum commene, Coriolus versicolor and Lentinus edodes is sold as a medicine such as an anticancer (See Non patent documents 1 and 2).

On the other hand, Aureobasidium sp. belonging to deuteromycetes is known to produce extracellularly β-glucan.

It is reported that the microorganism simultaneously produces β-1,3-1,6-D-glucan as well as fructooligosuccharide (Patent document 1) and produces β-glucans as well as pullulan (Patent document 2).

Aureobasidium sp. K-1 produces exocellularly β-1,3-1,6-D-glucan in the culture medium containing sucrose as a carbon source (See Non patent documents 3 and 4) and its structure consists of β-1,3-D-glucan as a main chain which binds at β-1,6-binding to D-glucose as a side chain and a part of the glucose residue as a side chain is substituted with HO₃SCH₂COOH residue, and the molecular weight of this polysaccharaides is estimated as 2 millions by gel filtration method (GPC) (Non patent documents 3 and 4, Patent document 3).

It is reported that β-1,3-1,6-D-glucan produced by a microorganism belonging to Aureobasidium sp. has also immunomodulatory activity, and this sugar polymer is useful as a functional food (enhancement of Bifidobacterium in intestinal tract, anticonstipation, immunomodulation) and a medicine for intestinal disorder (See Patent documents 2, 4 and 5).

Generally, an aqueous β-glucan solution is easily apt to form a single helical structure or a triple helical structure to form gel. Therefore, a culture medium of β-glucan is highly viscous, and it was very difficult to purify from the medium (See Non patent document 1). As well, β-glucan produced by a microorganism belonging to Aureobasidium sp. is highly viscous and there are a few methods to separate, recovery and purify β-glucan in the industrial scale.

The methods reported now are as follows:
(1) In Patent document 1, it is disclosed that by sterilization of the culture medium of Aureobasidium sp. FERM-P. No. 4257 under heating and by filtration or centrifugation of the medium, there is obtained a culture medium usable as a drink or a food.
(2) In Patent document 6, it is disclosed that by alkali-treating insoluble β-glucan under heating in the presence of an organic solvent, there is recovered the insoluble β-glucan.
(3) In Patent document 7, it is disclosed that to a cell-suspension of Lentinus edodes or a microorganism, are added a peroxide and a hydroxy compound, and the mixture was adjusted pH to 10-12, followed by recovery and purification of the insoluble β-glucan.
(4) In Patent document 8, it is disclosed that after sterilizing the culture medium under heating, the cells are removed by centrifugation, and to its supernatant is added ethanol so as to be more than 60% (v/v) to precipitate β-glucan and then purifying by filtration, recovering and drying to get β-glucan.

However, in regard to the method of the above (1), the culture medium containing β-1,3-1,6-D-glucan in its high concentration is highly viscous (more than several thousand cP ([mPa.s])), and the separation of the insoluble materials such as cells from the culture medium by filtration or centrifugation is difficult in the industrial scale.

The methods of the above (2) to (4) relate to the method for isolation or recovery of insoluble β-glucan present mainly in cell wall of a microorganism, such as Lentinus edodes or yeast, and β-D-glucan obtained by the method described in these (2) to (4) is hardly insoluble in water, is colored and therefore, there are problems to be used as materials of foods, especially materials of soft drinks.

Therefore, it was recently reported that after extracting with an alkali or hot water and partially purification of an insoluble β-glucan obtained from Lentinus edodes, etc., the glucan was treated under high pressure (300-800 kgf/cm²) in the presence of a usual dispersing agent containing an emulsifying agent such as lecithin as a main component, to make super micronization in colloidal form (See Patent document 9). However, this method requires a special micronizing procedure and an emulsifier.

As an aqueous β-glucan solution is highly viscous, the solution is obtainable only in low concentration and therefore, there are many problems accompanying it. The development of its solution in high concentration has been desired.

Furthermore, the development of purified β-glucan in powder has been also desired, but as it is difficult to remove the cells, the method for preparing the powder containing the cells, culture medium, auxiliaries for spray drying, and an alkali, etc., in low concentration is only known. In regard to the method by crystallizing β-glucan from an aqueous ethanol solution (Patent document 8), the concentration of it is low and the method is not said to be practical from the view point of the industrial production.

Patent document 1: Japanese Patent Publication A 61-146192

Patent document 2: Japanese Patent Publication A62-201901

Patent document 3: Japanese Patent Publication A 7-51082

Patent document 4: Japanese Patent Publication A 6-340701

Patent document 5: Japanese Patent Publication A 2002-204687

Patent document 6: Japanese Patent Publication A 5-308987

Patent document 7: Japanese Patent Publication A 9-322795

Patent document 8: Japanese Patent Publication A 10-276740

Patent document 9: WO 02/087603

Non patent document 1: Fragrance Journal, 5, 71-75 (1995)

Non patent document 2: Nikkei Bio, No.2, page 91-94 (2003)

Non patent document 3: Agric. Biol. Chem., 47, 1167-1172 (1983)

Non patent document 4: Kagaku and Kogyo, 64, 131-135 (1990)

### DISCLOSURE OF INVENTION

The present inventors found a process for preparing a low viscous β-glucan solution from the culture medium of a microorganism belonging to Aureobasidium sp. and filed a patent application (Japanese Patent Appln. No. 2003-106676 (Japanese Patent Publication A 2004-092330)). Namely they developed the process for recovering an aqueous solution containing low viscous and aqueous β-1,3-1,6-D-glucan which is characterized in adding an alkali at room temperature to a culture medium containing, as a main ingredient, an aqueous β-1,3-1,6-D-glucan which a microorganism belonging to Aureobasidium sp. produces extracellularly, adjusting pH of the mixture to 12 or more to decrease its viscosity, adding an acid thereto to adjust pH from neutral (pH7.5) to acidic region (pH4 or less than pH4), and separating the insoluble cells to get a solution containing β-1,3-1,6-D-glucan in good yield.

The present inventors have further studied on the culture medium obtained by the above method developed by them (above Japanese Patent Publication A 2004-092330), and as a result they have found that β-1,3-1,6-D-glucan has a characteristic property in relation of its solubility with pH and temperature, its molecular weight and its particle size distribution, and that have continued to study on the improvement on the process thereof. As a result, it has been found that the preservative stability in the aqueous β-1,3-1,6-D-glucan solution (in respect of such as gelation, aggregation, etc.) and thermostability thereof are improved and further, the aqueous β-1,3-1,6-D-glucan solution has immunomodulatory property, etc. Thus the present invention has been completed. In addition, a process for effectively preparing β-1,3-1,6-D-glucan in powder with high purity has been found.

It has been found that β-1,3-1,6-D-glucan provided by the present invention is simultaneously co-existing in the form of a solution and in the form of micro pulverized dispersion, and its stability was greatly influenced by pH, metal ion (Na, Ca) strength and temperature. Much interestingly, in regard to the present microparticle β-1,3-1,6-D-glucan, it becomes clear that β-1,3-1,6-D-glucan beyond the range of its solubility is microcrystallized due to or in relation to the solubility determined by various conditions such as pH, metal ion (Na, Ca) strength, and temperature (See examples 5 and 6). The present microparticle β-1,3-1,6-D-glucan has been found to be derived from aqueous glucan having molecular weight 10 thousands to 500 thousands.

The present microparticle β-1,3-1,6-D-glucan aggregates and further gels at 35° C. or more in much metal ions. Therefore, in case of use it in a liquid food such as a soft drink, when sterilization by heating, for example at pH3.5 at temperature of 90° C., heterogeneity due to aggregation and further gelation occurs. For use it as a soft drink, muddiness (cloud) and precipitate are not preferable. It has been found that these troubles are greatly solved by controlling the metal ion concentration thereof.

On the other hand, for use it in purpose for gelation or thickening, gelation and thickening can increase by increasing metal ion concentration within the use. One of the objects of the present invention is to provide an aqueous β-1,3-1,6-D-glucan solution which is controlled in thermostability and preservative stability.

The present invention relates to an aqueous β-1,3-1,6-D-glucan solution, wherein ¹HNMR spectrum on said solution containing 1N aqueous sodium hydroxide heavy solution have two signals of 4.7 ppm and 4.5 ppm, and the viscosity (BM type rotary-viscometer, 12 rpm) of said solution at 30° C., pH5.0, and its concentration 0.5% (w/v) is 50 cP ([mPa.s]) or less, and preferably 40 cP or less, more preferably 30 cP or less.

The present invention relates to the above β-1,3-1,6-D-glucan solution wherein its molecular weight is 10 thousands˜500 thousands; the above β-1,3-1,6-D-glucan solution wherein its primary particle size is 0.05˜2.0 μm; the above β-1,3-1,6-D-glucan solution wherein ratio of β-1,3 bond/β-1,6 bond of β-1,3-1,6-D-glucan is 0.5˜2.0; the above β-1,3-1,6-D-glucan solution wherein ratio of β-1,3 bond/β-1,6 bond is 1.0˜1.5 based on signal integral of β-1,3 bond/β-1,6 bond in ¹HNMR spectrum of the solvent of 1N aqueous sodium hydroxide heavy solution; the above β-1,3-1,6-D-glucan solution wherein the β-1,3-1,6-D-glucan has a group containing sulfur; the above β-1,3-1,6-D-glucan solution wherein β-1,3-1,6-D-glucan is produced out of the cell by a microorganism belonging to Aureobasidium sp.; the above β-1,3-1,6-D-glucan solution wherein the microorganism is Aureobasidium pullulans; the above β-1,3-1,6-D-glucan solution wherein the microorganism is Aureobasidium pullulans GM-NH-1A1 or Aereobasidium pullulans GM-NH-1A2; the above β-1,3-1,6-D-glucan solution wherein the microorganism is not contained; the above β-1,3-1,6-D-glucan solution wherein metal ion concentration, especially Na ion and Ca ion are 120 mg/100 ml or less; and the above β-1,3-1,6-D-glucan solution wherein the microparticle β-1,3-1,6-D-glucan is removed from the aqueous β-1,3-1,6-D-glucan solution.

The present invention also relates to the microparticle β-1,3-1,6-D-glucan which is prepared by removing water soluble β-1,3-1,6-D-glucan from above aqueous β-1,3-1,6-D-glucan solution; a solution containing β-1,3-1,6-D-glucan which is prepared by incorporating above aqueous β-1,3-1,6-D-glucan solution and the above microparticle β-1,3-1,6-D-glucan in desired amount; and a dried β-1,3-1,6-D-glucan prepared by drying the above aqueous β-1,3-1,6-D-glucan solution.

The present invention also relates to a process for preparing the above β-1,3-1,6-D-glucan solution which comprises adding an alkali or its aqueous solution to the microorganism-culture medium containing β-1,3-1,6-D-glucan, adjusting pH12 or more to make the medium lower viscosity, separating and removing the insoluble materials including a microorganism, and further removing the metal ions, especially Na ion and Ca ion so that the metal ion concentration becomes 120 mg/ 100 ml or less; a process for preparing β-1,3-1,6-D-glucan in powder with high purity which comprises dialyzing and concentrating above aqueous β-1,3-1,6-D-glucan solution until the concentration of β-1,3-1,6-D-glucan becomes 1.0% (w/w) or more, by adding an alcohol to its concentrate to precipitate β-1,3-1,6-D-glucan and then, separating thus obtained a slurry consisting of β-1,3-1,6-D-glucan/alcohol/water by a separating funnel and spray-drying the precipitated slurry-D-glucan; and a process for preparing β-1,3-1,6-D-glucan in powder with high purity which comprises by dialyzing the above aqueous β-1,3-1,6-D-glucan solution, further concentrating in vacuo and then spray-drying the concentrated solution.

Furthermore, the present invention relates to an antimalignancy neoplasm agent, especially an antimallignant sarcoma agent, an anticancer, an INF-γ-inducing agent, a therapeutic or prophylactic agent for diseases caused by INF-γ induction, an inhibiting agent for metastasis of malignancy neoplasm, an antiallergic agent, especially an antiallergic agent for type I allergic disease, and a macrophage activating agent containing the aqueous β-1,3-1,6-D-glucan solution or β-1,3-1,6-D-glucan as an active ingredient.

The present invention also relates to a healthy food or a health promoting agent, an external agent and a cosmetic containing the aqueous β-1,3-1,6-D-glucan solution or β-1,3-1,6-D-glucan, too.

The microorganism used in the present invention is not limited as far as the microorganism produced β-1,3-1,6-D-glucan, preferably the microorganism belonging to Aureobasidium sp.

Several methods for preparing β-1,3-1,6-D-glucan (sometimes abbreviated as glucan) by cultivating the microorganism belonging to Aureobasidium sp. are reported. As a carbon source used in these methods, a carbohydrate such as sucrose, glucose, fructose, etc. and an organic nutrient such as peptone, yeast extract, etc. are illustrated.

As a nitrogen source, an inorganic nitrogen source such as ammonium sulfate, sodium nitrate, potassium nitrate, etc. is illustrated. If necessary in order to raise the production of said β-glucan, an inorganic salt such as sodium chloride, potassium chloride, phosphates, magnesium salts, calcium salts, etc. and furthermore small amount of a metal salt such as Fe, Cu, Mn or vitamins may be preferably added thereto.

For example, it is reported that when a microorganism belonging to Aureobasidium sp. is cultivated in a Czapek medium containing sucrose as a carbon source and containing ascorbic acid, β-1,3-1,6-D-glucan is produced in high concentration (Non patent documents 3 and 4, Patent document 3). However the medium is not limited as far as the medium in which the microorganism grows and produces β-1,3-1,6-D-glucan. If necessary, an organic nutrient such as yeast extract or peptone may be added thereto.

The microorganism belonging to Aureobasidium sp. is aerobically cultured in the above culture medium at 10-45° C., preferably 20-35° C., at pH3-7, preferably 3.5-5.

To effectively control pH in the culture medium, it is a better procedure to control pH by an alkali or an acid. In addition, to prevent foam an anti-foam agent may be added thereto. The cultivation is preferably for 1 to 10 days. When the cultivation is usually for 1 to 4 days, β-glucan can be obtainable. The cultivation may be done during measuring the produced amount of β-glucan.

When the microorganism belonging to Aureobasidium sp. was aerobically cultivated under agitating in the above conditions for 4 to 6 days, β-glucan polysaccharide from 0.1% to several % (w/v) containing β-1,3-1,6-D-glucan as a main ingredient contained in the culture medium, and the culture medium has a very high viscosity several hundreds cP ([mPa.s]) to several thousands cP ([mPa.s]) at 30° C. by (BM type rotary viscometer (by Tokimec Inc.)).

When an alkali to this culture medium is added under stirring at room temperature, the viscosity thereof rapidly decreases. The alkali used therein is not limited as far as the alkali can be dissolved in water. In case of sodium hydroxide, it is added so that the final concentration becomes 0.5% (w/v) or more, preferably 1.25% (w/v) or more and the mixture is required to be well stirred. Namely, by adding an alkali to adjust pH 12 or more, preferably 13 or more and by stirring the mixture, the viscosity of the culture medium at once decreases to only several cP ([mPa.s]).

The method to separate cells from the alkali-treated culture medium is not limited as far as the method for separating cells from the culture medium such as the method for recovering the supernatant after cells precipitate (Decant method), the centrifugation method, the filtration method by using filter paper or filter cloth, the filter press method, the membrane filtration method (such as MF membrane), etc. In case of the whole filtration method by filter paper or filter cloth, the filtration auxiliary such as celite pad can be utilized. The cell-removal by filter press is preferable from the view point of the industrial production.

Before removal of the insoluble materials such as cells, pH may be adjusted to neutral or acidic by adding an acid or after separation of them, pH may be adjusted to neutral or acidic. Even pH of the medium is readjusted to neutral or acidic at room temperature (15˜35° C.), there is no possibility of gelation of β-glucan polysaccharide, and the viscosity is not changed without becoming highly viscous. As such the present invention relates to the aqueous β-1,3-1,6-D-glucan solution useful for the materials for healthy food and cosmetics, as the viscosity is not changed even by alkali-treating the aqueous highly viscous β-glucan solution to be lower viscous and subsequently readjusting pH to acidic (pH4>).

Preparations of healthy soft drinks are illustrated below.

1) Soft drink A

| Ingredient | Amount (weight %) |
|---|---|
| Aqua β* | 99.85 |
| Acesulfam | 0.10 |
| Lemon essence | 0.05 |

*Aqueous lower viscous β-1,3-1,6-D-glucan solution prepared by DAISO CO., LTD.

2) Soft drink B

| Ingredient | Amount (weight %) |
|---|---|
| Aqua β* | 50 |
| Aloe extract | 50 |

3) Soft drink C

| Ingredient | Amount (weight %) |
|---|---|
| Aqua β* | 40 |
| Aloe extract | 20 |
| Vital K (Tonic vegetable essence) | 40 |

A preparation of cosmetic is illustrated below.

| Ingredient | Amount (weight %) |
|---|---|
| Aqua β* | 80 |
| Sericin | 0.3 |
| 1,3-Butylene glycol | 5 |
| Glycerin | 2 |
| Chamomilla redutita extract | 2 |
| Sea wood extract (Sodium alginate) | 2 |
| Ascorbic acid sodium phosphate | 1 |
| Methylparaben | 0.2 |
| Water | 7.5% |

The alkali used for alkali-treatment includes an aqueous alkali carbonate solution such as an aqueous sodium carbonate solution, an aqueous potassium carbonate solution, an aqueous sodium carbonate solution, an aqueous ammonium carbonate solution; an aqueous alkali hydroxide solution such as an aqueous sodium hydroxide solution, an aqueous potassium hydroxide solution, an aqueous calcium hydroxide solution; or ammonia solution. In case of use of β-1,3-1,6-D-glucan as foods, the alkalis approved as food additives are preferably used.

The acid used for neutralization or acidification includes hydrochloric acid, phosphoric acid, sulfuric acid, citric acid, and malic acid. In case of use of β-1,3-1,6-D-glucan for foods, the acids approved as food additives are preferably used.

The culture medium treated by an alkali, an acid or treated neutrally itself may be served as foods, but in considering of sterilization or preserve-stabilization later, it is preferable to remove metal ions.

The metal ions are removed by desaltation by using UF membrane. The concentration of the metal ion is less than 120 mg/100 ml medium, preferably less than 50 mg/100 ml, and more preferably 20 mg/100 ml. For example, when making lower viscosity by alkali-treating with sodium hydroxide, the said metal ion is sodium ion.

The result on thermostability test using the medium of Example 1 is shown the following Table 1.

The culture mediums in which Na ion concentration was adjusted by buffer or sodium chloride were allowed to stand in an incubator at 50° C., and their stabilities were checked. The concentration of polysaccharides was adjusted to 2 mg/ml, and the concentration of Ca ion was adjusted to 10 mg/100 ml.

The signals in Table 1 are as follows:

∘: no change, x: gelation, aggregation, or precipitation

Gelation means lost of fluidization herein.

TABLE 1
Preservative stability at 50° C. (Effect of Na ion)

| Concentration | | | | | |
|---|---|---|---|---|---|
| of Na ion | After | After | After | After | After |
| [mg/100 ml] | 1 day | 2 days | 5 days | 10 days | 15 days |
| 19 | ∘ | ∘ | ∘ | ∘ | ∘ |
| 40 | ∘ | ∘ | ∘ | ∘ | ∘ |
| 60 | ∘ | ∘ | ∘ | x | x |
| 84 | ∘ | ∘ | ∘ | x | x |
| 112 | ∘ | ∘ | x | x | x |
| 160 | x | x | x | x | x |
| 260 | x | x | x | x | x |

The same test on Ca ion was carried out in the same manner as mentioned above. The result was shown in Table 2.

The culture medium of Example 1 was desalted by UF membrane and Ca ion concentration was adjusted by calcium chloride. The cultures were allowed to stand in an incubator at 50° C., and their stabilities were checked as well. The concentration of polysaccharides was adjusted to 2 mg/ml, and the concentration of Na ion was adjusted to 16 mg/100 ml.

∘: no change, x: gelation, aggregation, or precipitation

Gelation means a loss of fluidization herein.

TABLE 2
Preservative stability at 50° C. (Effect of Ca ion)

| Concentration | | | | | |
|---|---|---|---|---|---|
| of Ca ion | After | After | After | After | After |
| [mg/100 ml] | 1 day | 2 days | 5 days | 10 days | 15 days |
| 2 | ∘ | ∘ | ∘ | ∘ | ∘ |
| 31 | ∘ | ∘ | ∘ | ∘ | ∘ |
| 63 | ∘ | ∘ | ∘ | x | x |
| 84 | ∘ | ∘ | ∘ | x | x |
| 122 | ∘ | ∘ | x | x | x |
| 227 | x | x | x | x | x |

The recovery and purification method of β-1,3-1,6-D-glucan can be applicable to β-glucan from high molecular weight (2 millions or more) to lower molecular weight (several hundreds to 2 thousands) produced mainly by any strain of a microorganism belonging to Aureobasidium sp., and also applicable to β-1,3-1,6-D-glucan produced by all other microorganisms, or Lentinus edodes. Aureobasidium sp. namely Aureobasidium sp. K-1, Aureobasidium pullulans GM-NH-1A1 and Aureobasidium pullulans GM-NH-1A2 are especially preferable. It is known that the original strain, namely Aureobasidium sp. K-1 produces several kinds of high molecular weight β-glucan (molecular weights: 2 millions or more and about 1 million). Furthermore, it is known that β-glucan produced by Aureobasidium sp. K-1 has sulfoacetic acid group (See Non patent documents 3 and 4).

The mutant strains, GM-NH-1A1 and GM-NH-1A2 produce two kinds of β-glucans having an apparent main peak of 500 thousands˜2.5 millions higher molecular weight β-glucan (microparticle glucan) and an apparent main peak of 20 thousands˜300 thousands lower molecular weight β-glucan as show in Examples. The properties of these two strains, GM-NH-1A1 strain and GM-NH-1A12 strain are shown in Table 3 below. Judging from morphological properties on each strain and base sequences of 28S-rDNA, these strains are identified to be strains belonging to Aureobasidium pullulans. These strains have been deposited at International Patent Organism Depositary National Institute of Advanced Industrial Science and Technology (National Institute of Bioscience and Human-Technology Agency of Industrial Science and Technology) as FERM BP-10294 and FERM BP-10295, respectively.

Judging from the measure on particle distribution of the present culture medium, it has been found that in the culture medium solution, β-1,3-1,6-D-glucan is not completely dissolved, but in part, the β-1,3-1,6-D-glucan presents in the form of particles having the primary particle having the particle size of about 0.05˜2.0 μm.

Furthermore, by adding to the solution containing this fine particle β-1,3-1,6-D-glucan a known emulsifying agent such as lecithin, etc. or a stabilizer such as cyclic dextrin, etc., the solution can be further stabilized.

TABLE 3
Morphological property of GM-NH-1A1 and GM-NH-1A2

| Item | Property |
|---|---|
| Speed of colony | 2% MEA medium, Size of colonies; 42-48 mm |
| growth | |
| (25° C., 7 days) | |
| | PCA medium, Size of colonies; 34-35 mm |
| Color of colonies | 2% MEA medium; yellowish gray to olive color |
| Tissue of colony | Yeastlike |
| Hypa | Colorless to dark brown, Smooth, Dark brawn |
| | cell wall becoming thick. Breadth of colorless |
| | hypa: 2-5 μm, Breadth of dark brown hypa: |
| | 2-10 μm |
| Conidiogenous | Colorless, Smooth, Thin cell wall. Condium is |
| cell | formed directly on hypa or from denticle |
| | projection formed at side of hypa. |
| Conidium | Colorless, Smooth, Ellipse to cylinder 5-8 μm × |
| | 2-3 μm |
| Endospore | Colorless, Smooth, Ellipse to cylinder 4-6 μm × |
| | about 2 μm |

2% MEA: 2% multo extract agar plate medium
PCA: potato carrot agar plate medium

Dried β-1,3-1,6-D-glucan is prepared by subjecting an aqueous lower viscous β-1,3-1,6-D-glucan solution to a known drying method such as splay dry method, freeze-dry method and so on. In this method the lower viscous β-1,3-1,6-D-glucan solution may contain cells or salts.

The present invention related to a process for preparing β-1,3-1,6-D-glucan in powder which comprises adding an alkali or its aqueous solution to a microorganism-culture medium containing β-1,3-1,6-D-glucan to make the medium lower viscosity and if necessary, separating and removing the insoluble materials including cells of a microorganism, and by dialyzing and concentrating the above aqueous β-1,3-1,6-D-glucan solution until the concentration of β-1,3-1,6-D-glucan becomes 1.0% (w/w) or more, adding an alcohol to its concentrate under stirring to precipitate β-1,3-1,6-D-glucan and then, separating thus obtained slurry consisting of β-1,3-1,6-D-glucan/alcohol/water by a separating funnel and adjusting the concentration of the precipitated slurry so as to become 1.0% (w/w) or more, and spray-drying the precipitated slurry-D-glucan.

According to this method, as the volume of the lower viscous β-1,3-1,6-D-glucan solution can become several times to several ten times lower by concentration, the volume of an alcohol used for precipitating the polysaccharide can be remarkably reduced. Furthermore, according to the present method, in case of addition of the alcohol, as high molecular weight β-1,3-1,6-D-glucan dominantly precipitates and inorganic salts such as sodium citrate can be removed, the method is very useful for purity up and purification thereof.

The microorganism culture is preferably one containing as a main ingredient, β-glucan, especially β-1,3-1,6-D-glucan which a microorganism belonging to Aureobasidium sp. produces out of its cell. The separation and removal of the microorganism is preferably carried out by filter press or centrifugation. The alcohol is not limited as far as the alcohol can precipitate β-1,3-1,6-D-glucan from the culture medium, and ethanol is especially preferable. The volumetric amount of ethanol is the same volume or more, preferably two times or more than that of the culture medium. After separation by a separating funnel, the alcohol is recovered by distillation from the mixture of the alcohol and water and the alcohol can be used again (served as recycle).

On the other hand, the lower viscous β-1,3-1,6-D-glucan in powder can be produced without using of the alcohol as mentioned below. Namely, it has been found that β-1,3-1,6-D-glucan in powder can be produced after making the viscous lower by adding alkali or its solution to a cell culture medium containing β-1,3-1,6-D-glucan and if necessary, removing the insoluble materials containing the microorganism and then, if necessary dialyzing and concentrating well, by concentrating in vacuo and then by spray drying. By this method, the loss of the alcohol is little and the equipment for anti-explosion is not necessary and β-1,3-1,6-D-glucan in powder can be cheaply obtained with high purity. The process for preparing β-1,3-1,6-D-glucan in powder comprising such a condensing step under reduced pressure becomes possible because the aqueous β-1,3-1,6-D-glucan solution is lower viscous.

The aqueous β-1,3-1,6-D-glucan solution of the present invention, as shown in Examples, shows anti-malignancy neoplasm activity and antimetastasis activity of malignancy neoplasm in the test on malignancy neoplasm inhibiting test using colon cancer cells of mouse, Colon 26.

Therefore, β-1,3-1,6-D-glucan or its solution of the present invention is useful as an anti-neoplasm agent such as an anticancer, etc.

Furthermore, it has been found that the number of NK (natural killer) positive cells in the mucosal tissue of small intestine and the number of INF-γpositive cells significantly increased comparing with the non β-glucan-administration group, and IL-12 production in blood increases, too. This suggest that the present β-1,3-1,6-D-glucan activates the macrophages which are an antigen-presenting cell.

Therefore, the β-1,3-1,6-D-glucan or its solution of the present invention is expected as following agents:

1. An Antiviral Agent and an Antibacterial Agent

INF-γ activates RNAase (RNA decomposing enzyme) in cells and further prevents the synthesis of DNAs, proteins or peptides, and as a result, the proliferation of virus is inhibited. For example, inhibition of hepatitis A, B and C, hepatocirrhosis, herpes diseases, viral diseases such as influenza•gingivostomatitis, a various malignancy neoplasm (including the disease due to virus) and inhibition of metastasis thereof are observed. In addition, it is effective against infectious diseases such as MRSA•VRE, etc.

2. Type I Allergy (Anaphylaxy) Inhibitor

INF-γ inhibits the differential and activation of a kind of helper T cells, Th2 cell related to production of IgE antibodies. It is reported that INF-γ inhibits the production of IL-4. From the result, it is considered to inhibit allergy (Type I allergy) which IgE antibody is concerned. For example, it is expected to prevent pollinosis, allergic nasitis, allergic conjunctivitis, food allergy (gastrointestinal allergy such as hives or diarrhea), atopic dermatosis, allergic asthma, apisination, shock due to drugs such as penicillin, etc.

3. Antiallergic Agents (Type IV) and Antiautoimmune Disease Agent

As mentioned above 2, the self-(auto) substance partially denatured due to viral or bacterial infection becomes to have antigenicity (autoantigen), and it leads to autoimmune disease. For example, chronic arthrorheumatism, rheumatic fiver, collagen disease, Sjogren's syndrome, Hashimoto's disease, Basedow's disease, autoimmune hemolytic anemia, pernicious anemia, diabetes mellitus (type I), and serious adynamia are illustrated.

β-1,3-1,6-D-glucan or its solution is preferably orally administered. The dosage depends on the condition of the disease, age, body weight, administration times, etc. but is usually 1 to 200 mg (as β-1,3-1,6-D-glucan), preferably 5 to 100 mg.

### BRIEF DESCRIPTION OF THE DRAWINGS

FIG. 1: Two dimensional NMR (¹³C—¹H COSY NMR) spectrum FIG. 2: Particle distribution after radiation of ultrasonic waves (median size, 0.23 μm)

FIG. 3: Relationship with solubility and pH (concentration of polysaccharide, 0.2% at 25° C.)

FIG. 4: Relationship with solubility and temperature (concentration of polysaccharide, 0.2% at pH4)

### EXAMPLE

The present invention is explained in detail by the following examples, but should not be limited by these examples.

### Example 1

Use of Aureobasidium pullulans GM-NH-1A1

1) Production of β-glucan by Gultivation 1

The liquid culture medium (100 ml) consisting of the following ingredients shown in Table 4 was added into a 500 ml-flask with shoulders (shake flask), and after the medium was sterilized (autoclaved) at 121° C. for 15 minutes under vapor pressure, a loopful of Aureobasidium pullulans GM-NH-1A1 was aseptically inoculated from the slant medium (agar medium) and aerobically cultured for 24 hours at 30° C. at 130 rpm to prepare a seed culture medium. Then the same culture medium (200 L) as mentioned above was added to a 300 L-volume fermentor (by B. E. Marubishi Co., Ltd.) and sterilized (autoclaved) at 121° C., for 15 minutes under vapor pressure (0.15 Mpa). Then the previously prepared seed culture medium (2 L) was aseptically planted thereto, and the medium was aerobically cultured at 200 rpm at 27° C., aeration rate, 40 L/min. The medium pH was adjusted with sodium hydroxide and hydrochloric acid in the range of from 4.2 to 4.5. After 96 hours the turbidity of cells was 23 OD at OD 660 nm, and the content of polysaccharide was 0.5% (w/v).

By sampling the medium (several ml), removal of cells by centrifugation, adding ethanol to the supernatant so that the final concentration became 66% (v/v), precipitating the polysaccharide, recovering it, and dissolving it into ion-exchange water, the concentration of the polysaccharide was quantitatively analyzed by the phenol-sulfuric acid method.

Furthermore, to the cell-removed supernatant was added ethanol so that the final concentration became 66%, and β-glucan was recovered and rinsed once with 66% (v/v) ethanol. Then, the glucan was again dissolved in ion-exchange water, and centrifuged and then, to the supernatant was added brine so that the final concentration became 0.9%. Then, β-glucan was recovered with 66% ethanol. These β-glucan-recovery procedures were repeated twice, and thus obtained aqueous β-glucan solution was dialyzed and freeze-dried to give β-glucan n powder. From the result of analysis the present β-glucan, S (sulfur) content therein was 239 mg/kg and on calculation based on this, the amount of the substituted sulfoacetic acid was 0.09%.

TABLE 4
Ingredient of culture medium

| Compound | Concentration % (w/v) |
|---|---|
| Sodium nitrate | 0.2 |
| Potassium hydrogen phosphate | 0.1 |
| Potassium chloride | 0.1 |
| Magnesium sulfate.7H₂O | 0.05 |
| Ferrous sulfate.7H₂O | 0.001 |
| Ascorbic acid | 0.3 |
| Sucrose | 3 |
| pH 4.3 | |

2) Alkali Treatment

The viscosity of the culture medium obtained by Example 1 was measured by a BM rotary viscometer (Tokimec Inc.) at 30° C., at 12 rpm and the viscosity was 1500 cP ([mPa.s]). The rotor was chosen in accordance with the viscosity measured. To this medium was added 25% (w/w) sodium hydroxide so that the final concentration became 2.4% (w/v) and the mixture was stirred (pH 13.6). The viscosity was at once decreased and then, the solution was neutralized with 50% (w/v) citric acid to pH5.0. The viscosity was 20 cP ([mPa.s]) at 30° C. To the medium was added KCflock (by Nippon Paper Chemicals Co., Ltd.) 1wt % as a filter aid and by Yabuta-Compressing filter by Hagita Kikai) was removed the cells to finally get culture filtrate (about 230 L). The concentration of the polysaccharide was 0.5% (w/v) and the recovered rate was almost 100%.

3) Desaltation of the Aqueous β-glucan Solution

The above aqueous β-glucan solution (culture filtrate) was diluted so as to be 0.3% and the solution was desalted with UF membrane (Molecular weight cut 50 thousands, by Nitto Denko), so that sodium ion concentration finally became 20 mg/100 ml, and then pH was adjusted to 3.5 with 50% (w/v) aqueous citric acid solution. The solution was sterilized in a plate type liquid sterilizer (by Hisaka Works, Ltd.) at 95° C. for 3 minutes to get the final product, the aqueous β-glucan solution. The concentration of β-glucan was 0.22% (w/v) by measuring with phenol-sulfuric acid method. The total yield from the culture medium was about 73%. Thus obtained aqueous β-glucan solution was dialyzed with ion-exchange water and freeze-dried to give β-glucan in powder. As a result of analysis of the present β-glucan, S (sulfur) content therein was 330 mg/kg and on calculation based on this, the amount of the substituted sulfoacetic acid was 0.12%.

As the wave length on the above medium dialyzed was sifted from 480 nm to around 525 nm by Congo red method, it was confirmed that the solution contained glucan having β-1,3 bond (K. Ogawa, Carbohydrate Research, 67, 527-535 (1978), supervised by Tadayuki Imanaka, Great development of microorganisms, 1012-1015, N.T.S. (2002)).

The sift differences to maximum was Δ0.48/500 μg polysaccharide.

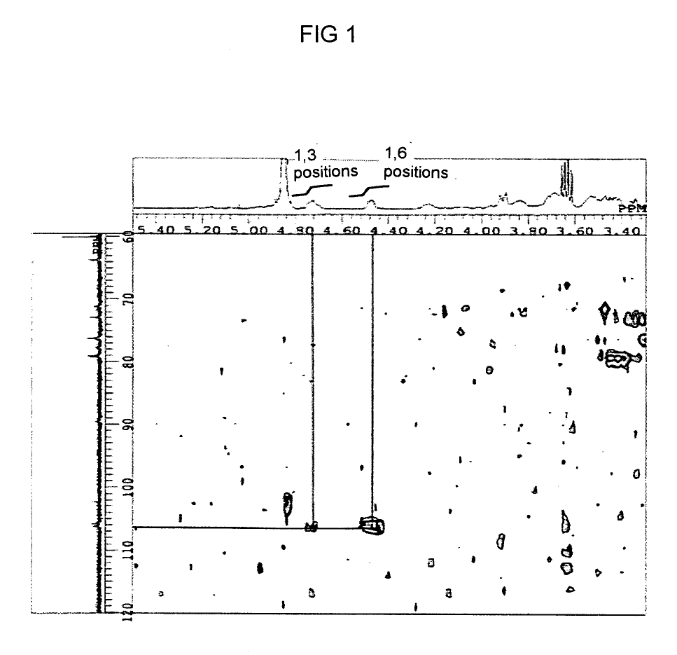

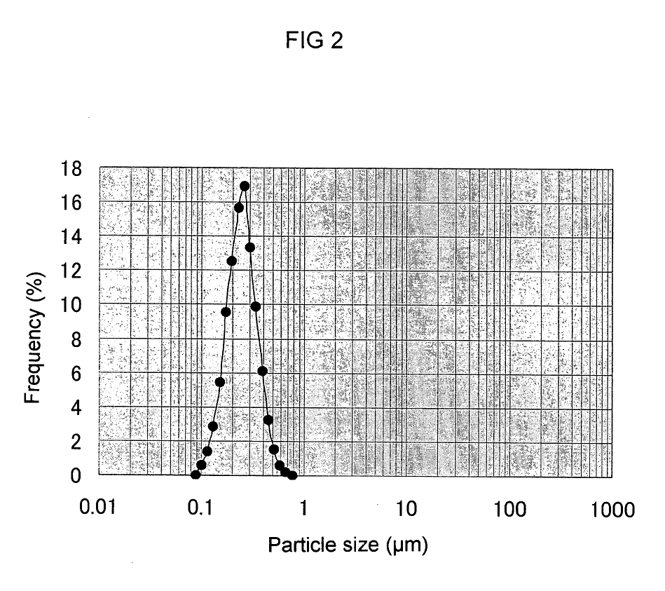

The above medium filtrate (15 ml) was taken out, and thereto was added ethanol 30 ml. The solution was centrifuged at 4° C. at 1000 rpm for 10 min. to get the precipitated polysaccharide. After washing with 66% ethanol, the polysaccharide was centrifuged at 4° C. at 1000 rpm for 10 min. To the precipitated polysaccharide were ion exchange water (2 ml) and 1N aqueous sodium hydroxide solution (1 ml) under stirring, and the precipitate was dissolved by keeping at 60° C., for 1 hour. The solution was freezed at −80° C., then dried in vacuo at room temperature over night and the powder was dissolved in heavy water (1 ml) to give two-dimensional NMR sample. Two signals at around 4.7 ppm and around 4.5 ppm of ¹HNMR spectrum having the correlation to the two-dimensional NMR (¹³C—¹H COSY NMR) 106 ppm as obtained (See FIG. 1). From this result, it was certified that the present β-glucan was β-1,3-1,6-D-glucan (supervised by Tadayuki Imanaka, Great development of microorganisms, 1012-1015, N.T.S., 2002). According to each integral ratio of ¹HNMR signal, it became clear that the ratio of β-1,3 bind/β-1,6 bind was 1.15.

When the above ¹HNMR spectrum was measured at 80° C. by using dimethyl sulfoxide d₆ heavy water solution in stead of 1N aqueous NaOH heavy water solution, two signals at around 4.7 ppm and around 4.5 ppm were sifted into 4.5 ppm and 4.2 ppm respectively.

Thus obtained β-1,3-glucan was hydrolyzed with exo-typed β-1,3-D-glucanase (kitalase M, K-I Chemical Industries Co., Ltd.) at 30° C. for several hours (See Patent document 3, and Non patent document 4). The hydrolyzed products was identified by HPLC analysis (acetonitrile/water, 70/30; 85° C.; flow 1 ml/min.) with Amido column (TSK-GEL AMIDE-80, diamiter 4.6 mm×250 mm, by Tosoh Corp.). Glucose and gentiobiose were identified as the hydrolyzed products. Each retention time was 5.7 min, (glucose) and 8.5 min. (gentiobiose). From this result, it was confirmed that the structure of glucan of the present invention consisted of β-1,3-D-glucan as a back bone which bound at β-1,6-binding to one molecule of D-glucose as a side chain.

Next, when the particle size in the culture solution was measured by using a laser spectrometry/scattering particle distribution meter (LA-920 by Horiba Ltd.), there were observed two peaks at around 0.3 μm and around 100 μm. Subsequently when ultrasonic wave was radiated thereto, the peak on 100 μm quickly disappeared and the peak on 0.3 μm increased and finally only the peak on 0.3 μm was remained (See FIG. 2). It was considered that the peak on 0.3 μm consisted of the primary particles of β-1,3-1,6-D-glucan and the peak on 100˜200 μm consisted of the secondary particles which was prepared by aggregation of the primary particles of β-1,3-1,6-D-glucan. Furthermore, it was confirmed that the secondary particle disappeared under stirring by a magnetic stirrer, or by slight agitation as well to become easily the primary particles. Therefore, the secondary particles were considered to be very mildly aggregated.

By using Toyopal HW65 by Tosoh Corp. (column size: 75 cm×φ1 cm, excluded molecular weight 2 millions and 500 thousands (dextran)) as elution solution of 0. 1M NaOH solution by subjecting to gel chromatography, the molecular weights of the dissolved β-1,3-1,6-D-glucan and the solution containing β-1,3-1,6-D-glucan consisting of the primary particles of β-1,3-1,6-D-glucan were measured. From the result, it became clear that the molecular weights of polysaccharide consisted of the lower molecular weight fraction having a peak of 2 to 300 thousands derived from dissolved β-1,3-1,6-D-glucan, and the apparent higher molecule weight fraction having a peak of 500 thousands to 3 millions derived from the primary particles of β-1,3-1,6-D-glucan. Pullulan (Shodex Company) was used as a marker of the molecular weight herein.

On the other hand, in order to separate the water soluble β-1,3-1,6-D-glucan and the particles, when the aqueous β-1,3-1,6-D-glucan solution (containing the particles and solubilized glucan) prepared by the method of the present invention was filtered by a filter (0.2 μm) (Advantec Co., Ltd.), the fraction of 500 thousands-2.5 millions molecular weight disappeared. Namely, it became clear that the higher molecular weight fraction corresponded to the secondary particles prepared by aggregation of the primary particles and the secondary particles of β-1,3-1,6-D-glucan. Therefore, the molecular weight of the aqueous β-1,3-1,6-D-glucan was considered to be 20˜300 thousands.

Next, the thermostability was investigated on sterilization under heating. In Example 1, after alkali-treatment, removal of cells and removal of metal ions to 50 mg/100 ml or less, the concentration of the polysaccharide was adjusted to 0.2% (2 mg/ml), 0.40%, 0.52%, 0.77%, and 0.96%, respectively, and they were subjected to heat treatment at 90° C. for 15 minutes for sterilization. The result was shown in Table 5.

∘: no change, Δ: partially aggregated, x: no fluidized gel

When the concentration of polysaccharide was high, the polysaccharide showed the tendency to apt to be aggregated. When the concentration of the polysaccharide was beyond 0.5% (5 mg/ml), it was apt to be aggregated and when the concentration of the polysaccharide was beyond 0.7%, it became non-fluidized gel.

TABLE 5
Stability on sterilization at 90° C.

| | Polysaccharide [%] | | | | |
|---|---|---|---|---|---|
| | 0.20 | 0.40 | 0.52 | 0.77 | 0.96 |
| Gelation | ∘ | ∘ | Δ | x | x |

### Example 2

Use of Aureobasidium sp. K-1

1) Production of β-glucan by Cultivation 2

The aqueous culture medium (60 ml) consisting of the ingredients shown in Table 4 was added into a 300 ml-Erlenmeyer flask, and after the medium was autoclaved and sterilized at 121° C. for 15 minutes, a loopful of Aureobasidium sp. K-1 in the slant medium was aseptically inoculated and aerobically cultured for 96 hours at 30° C. at 130 rpm to prepare a seed culture medium. After 96 hours the turbidity of cells was 35 OD at OD 660 nm, and the content of polysaccharide was 0.5% (w/v). To the cell-removed supernatant was added ethanol so that the final concentration became 66%, and β-glucan was recovered. Then, the glucan was again dissolved in ion-exchange water and centrifuged and then, to the supernatant was added brine so that the final concentration became 0.9%, and then, β-glucan was recovered with 66% ethanol. This β-glucan recovery procedures were repeated twice, thus obtained aqueous β-glucan solution was dialyzed, and freeze-dried to give β-glucan in powder. From a result of analysis of the present β-glucan, S content therein was 2300 mg/kg and by calculation based on this, the amount of the substituted sulfoacetic acid was 0.9%.

2) The Property on the polysaccharide was Tested in the Same Manner as in Example 1.

From the result of the investigation on the sift effect by congo red, the sifted from maximum absorption, 480 nm to around 525 nm was observed and it was confirmed that the solution contained glucan having β-1,3 bond. The sift differences to maximum was Δ0.50/500 μg polysaccharide.

According to each integral ratio of ¹HNMR signal, it became clear that the ratio of β-1,3 bind/β-1,6 bind was 1.38. Its molecular weight was 100 thousands to 3 millions.

The viscosity of the culture medium obtained was measured in the same manner as in Example 1 by a BM rotary viscometer (Tokimec Inc.) at 30° C. at 12 rpm, and the viscosity was 1900 cP ([mPa.s]). To this medium was added 25% (w/w) sodium hydroxide so that the final concentration became 2.4% (w/v) followed by stirring (pH13.4), the viscosity at once decreased. When the solution was neutralized with 50%(w/v) aqueous citric acid to pH 4.8, the viscosity was 28 cP ([mPa.s]) at that time.

### Example 3

Use of Aureobasidium pullulans GM-NH-1A2

By using Aureobasidium pullulans GM-NH-1A2 in stead of Aureobasidium pullulans GM-NH-1A1, the procedure was conducted in the same manner as in Example 1. As a result, the concentration of polysaccharide produced was 0.5% (w/v), and its viscosity was 1300 cP ([mPa.s]). In the same way as Example 2, to the medium prepared was added sodium hydroxide so that the final concentration became 2.4% (w/v) and the mixture was stirred (pH13.6). Then, the solution was neutralized with 50% (w/v) citric acid to pH5.0. By measuring the viscosity in the same way as in Example 1, it was 7 cP ([mPa.s]). The concentration of the polysaccharide was 0.5 (w/v) at that time.

The property on the polysaccharide was tested in the same manner as in Example 1.

As a result of the investigation on the sift effect by congo red, the sifted from maximum absorption, 480 nm to around 525 nm was observed. The sift differences to maximum was Δ0.45/500 μg polysaccharide. Furthermore, two dimensional NMR (¹³C—¹H COSY NMR) analysis was done. From these results, it was confirmed that the obtained product was β-1,3-1,6-glucan. According to each integral ratio of ¹HNMR signal, it became clear that the ratio of β-1,3 bind/β-1,6 bind was 1.23. It became clear that the molecular weights of polysaccharide consisted of the lower molecular weight fraction having a peak of 2 to 300 thousands and the higher molecule weight fraction having a peak of 500 thousands to 2.5 millions.

As a result of analysis of the β-glucan, S (sulfur) content therein was 229 mg/kg and by calculation based on this, the amount of the substituted sulfoacetic acid was 0.09%.

### Example 4

Change of Ingredients in Medium

Except changing the strain into Aureobasidium pullulans GM-NH-1A2 and without using ascorbic acid in Table 4, the procedure was carried out in the same manner as in Example 1. As a result, the concentration of the polysaccharide produced was 0.6%, and its viscosity was 1500 cP ([mPa.s]) at that time. In the same way as Example 2, to this medium obtained was added 25% (w/w) sodium hydroxide so that the final concentration became 2.4% (w/v) and the mixture was stirred (pH13.6). Then, the solution was neutralized with 50% (w/v) citric acid to pH 5.0 and the viscosity was measured in the same manner as in Example 1. The viscosity decreased to 7 cP ([mPa.s]).

From the result of the investigation on the sifting effect by Congo red, the sifted from maximum absorption, 480 nm to around 525 nm was observed. The sifting differences to maximum were Δ0.45/500 μg polysaccharide.

Furthermore, two dimensional NMR (¹³C—¹H COSY NMR) analysis was done in the same manner in Example 1. From these results, it was confirmed that the polysaccharide obtained was β-1,3-1,6-glucan. According to each integral ratio of ¹HNMR signal, it became clear that the ratio of β-1,3 bind/β-1,6 bind was 1.21. It became clear that as a result measuring the molecular weight by the same method of Example 3, the molecular weights of polysaccharide consisted of the lower molecular weight fraction having a peak of 2 to 300 thousands and the higher molecule weight fraction having a peak of 500 thousands to 2.5 millions.

Thus prepared β-1,3-1,6-D-glucan was analyzed in the same manner as in Example 1 and it was confirmed that the structure of glucan of the present invention consisted of β-1,3-glucan as a back bone which bound at β-1,6-binding to one molecule of D-glucose as a side chain.

### Example 5

Effect to Solubility by pH

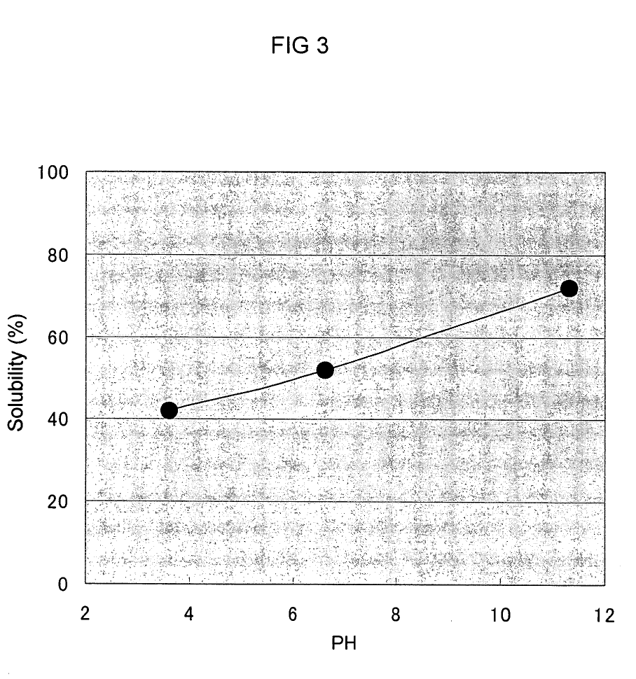

The solubility of β-1,3-1,6-D-glucan was changed in accordance with pH in the culture medium. As could be seen in Example 1, from a result of measurement of the particle dispersion, it was confirmed that a great part of β-1,3-1,6-D-glucan presenting in particles were 0.1 μm or more in size and therefore, it could be considered that the glucan which passed through a filter (0.2 μm) (Advantec Co., Ltd.) was dissolved and the glucan which did not passed through the filter were present as particles. Herein the presenting ratio (weight of polysaccharide in the medium passed through a filter (0.2 μm)/the weight of the polysaccharide in the medium×100 (%)) was defined as a solubility. The concentration of the polysaccharide in the medium of Example 1 was adjusted to 0.2% (2 mg/ml) and when its pH was changed, the solubility was shown in FIG. 3.

### Example 6

Effect to Solubility by Temperature

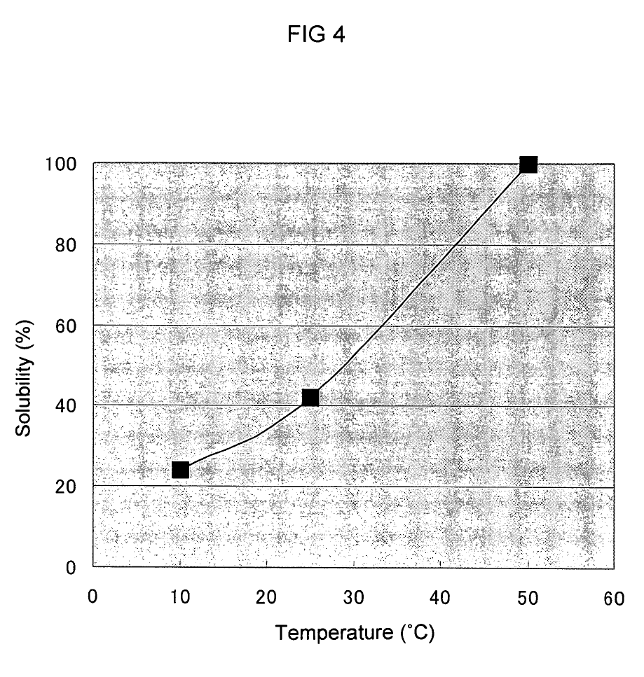

The solubility of β-1,3-1,6-D-glucan was greatly changed by temperature. Herein, by changing the temperature, and filtering by a filter (0.2 μm) (Advantec Co., Ltd.) in the same manner as in Example 5, the solubility was measured. After the concentration of the polysaccharide in the medium of Example 1 was adjusted to 0.2% (2 mg/ml), and when the temperature was changed, the solubility was shown in FIG. 4. Herein the presenting ratio (weight of polysaccharide in the medium passed through a filter (0.2 μm)/the weight of the polysaccharide in the medium×100(%)) was defined as a solubility.

As seen in Examples 5 and 6, by controlling pH and temperature it is possible to selectively prepare dissolved β-1,3-1,6-D-glucan, or particled β-1,3-1,6-D-glucan.

### Example 7

Property of Glucan in Powder

To the aqueous β-1,3-1,6-D-glucan solution containing particle β-1,3-1,6-D-glucan prepared by alkali-treating and cell-removal in Example 1 was added ethanol so as to be 66% (v/v) to precipitate polysaccharide glucan and it was recovered by centrifugation. Then, by removal of ethanol and water by freeze-dry, dried β-1,3-1,6-D-glucan was obtained. The yield was 95% or more comparing with the concentration of total saccharide before ethanol-precipitation. Then thus obtained dried β-1,3-1,6-D-glucan was dissolved and dispersed into water so that the final concentration became 0.3% (w/v), the solution, as shown in Example 1, was subjected to gel chromatography by Toyopal HW65 by Tosoh Ltd. (Column size 75 cm ×φ 1 cm, excluded m.w. 2 millions and 500 thousands (Dextran)) as elute; 0.1M sodium hydroxide solution, and the molecular weight was measured. As a result, it was confirmed that the molecular weight of the polysaccharide consisted of 2 peaks of a peak of a lower molecular weight fraction of 20 thousands˜300 thousands and a peak of apparent higher molecular weight of 500 thousands˜2 millions and 500 thousands. Pullulan prepared by Shodex Company was used as a marker of the molecular weight herein.

On the other hand, in order to separate the aqueous β-1,3-1,6-D-glucan and its particle, by filtrating the aqueous β-1,3-1,6-D-glucan solution (containing particle and solubilized glucan) prepared by the present method by a filter (0.2 μm) (Advantec Co., Ltd.), the higher molecular weight fraction of 500 thousands˜2 millions and 500 thousands molecular weight disappeared. Therefore, it was confirmed that by dissolving again glucan having the same physical property of the β-1,3-1,6-D-glucan before drying could be obtained, even if β-1,3-1,6-D-glucan prepared by the present invention was dried.

As explained above, it was found that lower viscous β-1,3-1,6-D-glucan could be obtained by alkali-treating a high viscous β-1,3-1,6-D-glucan produced by a microorganism belonging to Aureobasidium. β-1,3-1,6-D-glucan obtainable according to the method of the present invention contained an aqueous ingredient and a particle dispersed ingredient and the proportion of them could be controlled by pH and temperature. It was confirmed that by controlling metal ion concentration by desalting procedure, the culture solution excellent in the preservative and thermostabilities could be obtained. As a result, the present invention can provide the method for recovering β-1,3-1,6-D-glucan in high yield.

### Example 8

Preparation of β-1,3-1,6-D-glucan in Powder with High Purity

The culture medium solution treated by an alkali of Example 1 (the concentration of polysaccharide; 0.5% (5 mg/ml) (90 L)) was neutralized with aqueous 50% aqueous citric acid solution (9 kg) and then, cells were removed by Yabuta-Compressing filter 40D-4 precoated by filtration auxiliary (Cellulose powder KCflock by Nippon Paper Chemical Co., Ltd.) (1.8 kg) and the filtrate was concentrated to 9 L by ultrafilter with spiral element (NTU3150-S4 by Nitto Denko Corp.). To the concentrated solution was added under stirring ethanol (18 L) to get a slurry of glucan/ethanol/water. The viscosity of the slurry was 22 mPa.s (30° C.) by a BM viscometer. After being allowed to stand at room temperature for 3 hours, the supernatant (ethanol/water) (about 17 L) was removed. The viscosity of the residual slurry was 45 mPa.s (30° C.). The concentrated slurry (10 L) was spray-dried by a Sakamoto-Spray dry machine (Sakamoto Engineering Co., Ltd.) R-3 to obtain β-1,3-1,6-D-glucan in powder (360 g) (Recovery rate; 80%). The purity of thus obtained β-1,3-1,6-D-glucan was 90% or more as a result of NMR spectrum analysis.

The above obtained β-1,3-1,6-D-glucan contained about 10% of other polysaccharide than glucan, but it could be easily removed by washing with water.

### Example 9

The culture medium solution treated by alkali of Example 1 (the concentration of polysaccharide; (5 mg/ml)) (100 L) was neutralized with 50% aqueous citric acid solution (10 L) and then, cells were removed by Yabuta-Compressing filter 40D-4 precoated by filtration auxiliary (Nippon Paper Chemical, Co., Ltd., cellulose powder W-200) (1.8 kg) and the filtrate was concentrated to 9 L by the ultrafilter (NTU3150-S4 by Nitto Denko) to become a half volume (55 L). After concentration and addition of water were repeated, the solution was finally concentrated to 1/100. The solution was further concentrated to 5 L in vacuo. The viscosity of the concentrated solution was 100 mPa.s, and the concentration of the solid was 10%. The concentrated extract was dried by spray dry by Sakamoto-Spray dry machine R-3 (entrance temperature: 185° C., outlet temperature: 120° C. at 33000 rpm) to obtain β-1,3-1,6-D-glucan in powder (310 g) (Recovery rate; 62%). Thus obtained β-1,3-1,6-D-glucan 0.5% was dissolved in water and the concentration of the polysaccharide was 4.8 mg/ml. From this result, the purity of the powder was considered to be about 95% or more. The obtained powder was brown and its means particle size was 25 μm. Redispesion into water was good, and a 10% solution could be obtained by the simple stirring a 15% solution became paste and the paste could not be separated upside down of the vessel.

### Example 10

Malignancy Neoplasm Suppressive Activity and Immunomodulatory Activity (i. p.)

By using β-1,3-1,6-D-glucan (Abbreviated as glucan) prepared from Aureobasidium pullulans GM-NH-1A1 by Example 1, malignancy neoplasm suppressive activity and anti-malignancy neoplasm metastasis activity on mice were evaluated.

1. Method and Materials

1) Materials

Matrigel (Reduced Growth Factor) was purchased from Becton Dickison. Glucan prepared in Example 1 was used and dialyzed with physiological solution, removal cells by a filter membrane (0.45 μm), and then two glucan solutions (0.5 mg/ml and 1.5 mg/ml) were prepared to be subjected to the experiment. The solution was intraperitoneally (i.p.) administered to mice (0.1 ml/10 g/body weight).

2) Animal

Mice (Balb/c, male, 5 weeks old) were purchased from Clea Japan Inc. and after pre-feeding for 1 week, healthy mice among them were used.

3) Colon 26 Colon Cancer Cell

Colon 26 colon cancer cells, which were distributed by Institute of Development, Aging and Cancer, Tohoku Univ. and were subcultured, were used as malignancy neoplasm.

4) Transplantation of Colon 26 Colon Cancer Cells to Spleen of Mice

Colon 26 colon cancer cells (5×10⁴ cells/ml) were suspended in physiological phosphate buffer (PBS, pH7.4), and thereto was added Matrigel (1 mg/ml). The back of a mouse (Balb/c) was made small incision under Nembutal anesthesia. Spleen was exposed and therein was injected Colon 26 colon cancer cell-suspension (0.2 ml) (1×10⁴ cells/spleen). Then, the incision was sewn up. From the next day after Colon 26 colon cancer cells were transplanted, the prepared glucan solutions (5 mg/kg/body weight and 15 mg/kg/body weight) were intraperitoneally administered once a day for 14 days. Only a physiological saline solution was administered to the normal mouse group and the control mouse group. On the next day after the last day (on 15th day after transplantation) heparin was collected from the inferior vena cave under ether anesthesia. In addition, after collection of the blood, the mice were sacrificed, and spleen, liver and thymus were extracted therefrom, and the weight of each organ was measured. The number of colonies of Colon 26 colon cancer metastasized to liver was counted.

Furthermore, the small intestine extracted was fixed with 10% neutral formalin buffer. After wrapping with paraffin, the pathologic slice of the small intestine was prepared. After treating by the conventional method, the immune stain by using NK antibody and INF-γ antibody was carried out on the slice. The number of NK positive cells and INF-γ positive cells were counted.

Then, the amount of IL-12 in blood of the mouse with spleen transplanted by Colon 26 was measured with ELISA kit after centrifugation of the blood.

2. Result

1) Effect of glucan Administration (i.p.) to the Primary Cancer Cells of the Mouse in which Spleen was Transplanted with Colon 26 Colon Cancer

As shown in Table 6, when Colon 26 colon cancer cells were transplanted to spleen, the weight of cancer cells was clearly increased, but in the group of glucan-administration the increase of the weight of cancer cells was suppressed. Namely, in case of administration at 15 mg/kg, the suppression effect was 33.2%.

2) Effect of glucan Administration to the Metastasis to Liver of the Mouse in which Spleen was Transplanted with Colon 26 Colon Cancer

As shown in Table 6, when colon 26 colon cancer cells were transplanted to spleen, the metastasis to liver was clearly observed and the number of liver increased. On the other hand, in the glucan-intraperitoneal administration group, the number of colonies metastasized to liver was significantly suppressed comparing with the group of the mouse transplanted with Colon 26 colon cancer. Namely, in the administration group at 5 mg/kg, the suppression effect was 44.5%, and in case of the administration at 15 mg/kg, the suppression effect was 31.2%.

TABLE 6
Effect of glucan administration (i.p.) to the weight
of spleen primary cancer and the number of colonies
metastasized to liver of the mouse in which spleen
was transplanted with Colon 26 colon cancer

| | | Means ± standard error | |
|---|---|---|---|
| | | | Number of |
| | | | colonies |
| | Number | Primary cancer | metastasized |
| | of mice | weight (mg) | to liver |
| Normal group | 5 | 74.8 ± 3.29 * | |
| | | (Spleen weight) | |
| Colon 26 cancer cell | 8 | 1758.1 ± 278.1 | 154 ± 16 |
| transplanted mouse | | (100%) | (100%) |
| group | | | |
| + Glucan administration | 7 | 2079.0 ± 361.6 | 84 ± 23 * |
| (i.p.) group (5 mg/kg) | | (119.3%) | (54.5%) |
| + Glucan administration | 8 | 1174.0 ± 246.1 | 106 ± 22 * |
| (i.p.) group (15 mg/kg) | | (66.8%) | (68.8%) |

The significance assay was conducted by the multiple assay by Fisher's Protected LSD.
*: The significance to the group of the mouse which was transplanted with Colon 26 colon cancer cells was confirmed (P < 0.05).

3) The Effect of glucan Administration (i.p.) to the Intestinal Immune Function of the Mouse in which Spleen was Transplanted with Colon 26 Colon Cancer

As shown in Table 7, the number of NK positive cells in the mucosal membrane of small intestine significantly decreased with transplantation of Colon 26 colon cancer cells to spleen and in case of glucan intraperitoneal administrations at 5 mg and 15 mg/kg, the number of NK positive cells in the mucosal membrane of the small intestine significantly recovered to normal value. In case of glucan intraperitoneal administrations at 5 mg and 15 mg/kg, the number of INF-γ positive cells in the mucosal membrane significantly increased comparing with the number in Colon26 cancer cell-transplanted mice.

4) Effect of glucan Administration (i.p.) to the Amount of IL-12 in Serum of the Mouse in which Spleen was Transplanted with Colon 26 Colon Cancer

As shown in Table 8, when Colon 26 colon cancer cells were transplanted to spleen, the concentration of IL-12 in blood increased. It was considered that this fact showed that the immune mechanism was potentiated due to the biological defense so as to exclude the cancer. In glucan intraperitoneal administration at any dosage, the serum concentration of IL-12 increased comparing with the group of the mouse transplanted with Colon 26 colon cancer cells.

When glucan was intraperitoneally administered, the serum concentration of IL-12 increased and therefore, it was suggested that the glucan activated the macrophages which were antigen-presenting cells.

TABLE 7
Effect of glucan administration (i.p.) to the numbers
of NK and INF-γ positive cells in the mucosal membrane
of small intestine of the mouse in which spleen was
transplanted with Colon 26 cancer cells

| | | Means ± standard error | |
|---|---|---|---|
| | Number | Number of NK | Number of INF-γ |
| | of mice | positive cells/field | positive cells/field |
| Normal group | 5 | 248 ± 13 * | 222 ± 11 |
| Colon 26 cancer cell | 8 | 166 ± 13 | 221 ± 20 |
| transplanted mouse | | (100%) | (100%) |
| group | | | |
| + Glucan | 7 | 196 ± 15 | 274 ± 13 * |
| administration (i.p.) | | (118%) | (124%) |
| group (5 mg/kg) | | | |
| + Glucan | 8 | 212 ± 15 * | 265 ± 10 * |
| administration (i.p.) | | (128%) | (120%) |
| group (15 mg/kg) | | | |

The significance assay was conducted by the multiple assay by Fisher's Protected LSD.
*: The significance to the group of the mouse which was transplanted with Colon 26 colon cancer cells was confirmed (P < 0.05).

TABLE 8
The effect of glucan administration (i.p.) to the
amount of IL-12 in serum of the mouse in which spleen
was transplanted with Colon 26 colon cancer

| | Number | Mean ± standard error |
|---|---|---|
| | of mice | IL-12 (pg/ml) |
| Normal group | 5 | 587.1 ± 53.2 * |
| Colon 26 colon cancer | 8 | 1031.9 ± 115.2 |
| transplanted mouse group | | (100%) |
| + Glucan administration (i.p.) | 7 | 1321.8 ± 93.8 * |
| group (5 mg/kg) | | (128%) |
| + Glucan administration (i.p.) | 8 | 1494.8 ± 133.5 * |
| group (15 mg/kg) | | (145%) |

The significance assay was conducted by the multiple assay by Fisher's Protected LSD.
*: The significance to the group of mouse transplanted with Colon 26 colon cancer cells was confirmed (P < 0.05).

### Example 11

Malignancy Neoplasm Suppressive Activity and Immunomodulatory Activity (Oral Administration (p.o.))

By using β-1,3-1,6-D-glucan (abbreviated as glucan) prepared from Aureobasidium pullulans GM-NH-1A1 by Example 1, malignancy neoplasm suppressive activity and antimalignancy neoplasm metastasis activity were evaluated.

1. Method and Materials

Materials

1) Matrigel (Reduced Growth Factor) was Purchased from Becton Dickison Company. Glucan Prepared by Example 1 was Used and After Dialyzing it with Distilled Water, the glucan Solutions (5 mg/ml and 45 mg/ml) were Prepared to be Subjected to the Experiment. The Solution was Orally Administered to a Mouse (0.1 ml/10 g Body Weight).

2) Animal

Mice (Balb/c, male, 5 weeks old) were purchased from Clea Japan Inc. and after pre-feeding for a week, healthy mice among them were used for this test.

3) Colon 26 Colon Cancer Cell

Colon 26 colon cancer cells, which were distributed by Institute of development, Aging and Cancer, Tohoku Univ. and were subcultured, were used as malignancy neoplasm.

4) Transplantation of Colon 26 Colon Cancer Cells to Spleen of Mice

Colon 26 colon cancer cells (5×10⁴ cells/ml) were suspended in physiological phosphate buffer (PBS, pH7.4), and thereto was added Matrigel (1 mg/ml). The back of a mouse (Balb/c) was made small incision under Nembutal anesthesia. Spleen was exposed and therein was injected Colon 26 colon cancer cell-suspension (0.2 ml) (1×104 cells/spleen). Then, the incision was sewn up. From the next day after Colon 26 colon cancer cells were transplanted, the prepared glucan solutions (50 mg/kg/body weight and 450 mg/kg/body weight) were orally administered once a day for 14 days. Only a physiological saline solution was administered to the normal group and the control group. On the next day after the last day (on 15th day after transplantation) heparin was collected from the inferior vena cave under ether anesthesia. In addition, after collection of the blood, the mice were sacrificed, and spleen, liver and thymus were extracted, followed by measurement of each organ. The number of colonies of Colon 26 colon cancer cells metastasized to liver was counted.

Furthermore, the small intestine extracted was fixed with 10% neutral formalin buffer. After wrapping with paraffin, the pathologic slice of the small intestine was prepared. After treating by the conventional method, the immune stain by using NK antibody and INF-γ antibody was carried out on the slice. The number of NK positive cells and INF-γ positive cells were counted.

2. Result

1) Effect of glucan to Primary Cancer of the Mouse in which Spleen was Transplanted with Colon 26 Colon Cancer Cells

As shown in Table 9, when Colon 26 colon cancer cells were transplanted to spleen, the cancer cells clearly proliferated, but in the group of glucan-administration the increase of the weight of cancer cells was suppressed. Namely, in case of administrations at 50 mg/kg, the suppression effect was 27.9% and in case of administration at 450 mg/kg, the suppression effect was 23.9%, respectively.

2) Effect of glucan to Metastasis to Liver of the Mouse in which Spleen was Transplanted with Colon 26 Cancer Cells

As shown in Table 9, when colon 26 colon cancer cells were transplanted to spleen, the metastasis to liver was clearly observed and the weight of liver increased. On the other hand, in the glucan-oral administration group, the number of colonies metastasized to liver was significantly suppressed comparing with the group of the mouse transplanted with Colon 26 colon cancer. Namely, in the administration group at 50 mg/kg, the suppression effect was 37.3%, and in the administration group at 450 mg/kg, the suppression effect was 4.9%.

TABLE 9
Effect of glucan administration (p.o.) to the weight
of primary cancer of the mouse in which spleen was
transplanted with Colon 26 cancer cells and the number
of colonies of metastasized cancer to liver

| | | Means ± standard error | |
|---|---|---|---|
| | | | Number of |
| | | | colonies of |
| | Number | Primary cancer | metastasized |
| | of mice | weight (mg) | cancer to liver |
| Normal group | 5 | 73.0 ± 2.97 * | |
| | | (weight of spleen) | |
| Colon 26 colon cancer | 8 | 1809.3 ± 156.5 | 142 ± 11 |
| cell transplanted group | | (100%) | (100%) |
| + Glucan administration | 7 | 1305.3 ± 193.1 * | 89 ± 12 * |
| (p.o.) group (50 mg/Kg) | | (72.1%) | (62.7%) |
| + Glucan administration | 8 | 1376.3 ± 193.1 * | 135 ± 27 |
| (p.o.) group (450 mg/kg) | | (76.1%) | (95.1%) |

The significance assay was conducted by the multiple assay by Fisher's Protected LSD.
*: The significance to the group of the mouse which was transplanted with Colon 26 colon cancer cells was confirmed (P < 0.05).

3) Effect of glucan Administration (p.o.) to Intestinal Immune Function of the Mouse in which Spleen was Transplanted with Colon 26 Colon Cancer

As shown in Table 10, the number of INF-γ positive cells in the mucosal membrane of small intestine significantly decreased with transplantation of Colon 26 colon cancer cells to spleen and in case of glucan oral administration at 50 mg, the number of INF-γ positive cells in the mucosal membrane significantly increased comparing with the number in Colon 26 colon cancer cells transplanted mice. In the higher dose (450 mh/kg), the number of INF-γ positive cells in the mucosal membrane did not significantly increased comparing with the number in Colon 26 colon cancer cells transplanted mice.

TABLE 10
Effect of glucan administration (p.o.) to the number of INF-γ positive
cells in the mucosal membrane in small intestine of the mouse in which
spleen was transplanted with Colon 26 colon cancer cell

| | | Means ± standard error |
|---|---|---|
| | Number | Number of INF-γ |
| | of mouse | positive cell |
| Normal group | 5 | 331 ± 16 * |
| Colon 26 colon cancer cell | 8 | 281 ± 15 |
| transplanted mouse group | | (100%) |
| + Glucan administration | 7 | 333 ± 19 * |
| (p.o.) group (50 mg/kg) | | (119%) |
| + Glucan administration | 8 | 286 ± 17 |
| (p.o.) group (450 mg/kg) | | (102%) |

The significance assay was conducted by the multiple assay by Fisher's Protected LSD.
*: The significance to the group of the mouse which was transplanted with Colon 26 colon cancer cells was confirmed (P < 0.05).

### Example 12

Allergic Suppression Effect

1. Method

1) Allergic Immunization by Taking Orally Ovaalbumin

Mice (Balb/c, male, 4 weeks old) freely fed with powdered feed containing β-glucan (0, 0.25%, 0.5%, 1.0%) for test term (the β-glucose administration mouse group, the control group: the mouse fed with feed not containing β-glucan). For feeding, the amount of the consumed feed was every day measured on each cage (1 cage in 7 mice), and the body weight of the mouse was measured every one week. Two weeks later after feeding β-glucan, a 1% aqueous ovaalbumin solution was freely taken for a week (primary antibody production, primary immunization). Further, for the latter half, 3 days a 2.5% aqueous ovaalbumin solution was forced to take orally twice at morning and evening (ovaalbumin 5 mg×twice/mouse/day).

On 3^{rd} and 7^{th} days, after taking the aqueous ovaalbumin solution for the primary immunization, the physiological saline solution containing ovaalbumin (25 μg) and aluminum hydroxide gel (1.6 mg) was intraperitoneally administered to induce immunity (secondary antibody production, secondary immunization). On the next day after twice administrations of ovaalbumin was completed, serum was collected from the inferior vena cave under ether anesthesia. Spleen, thymus and small intestine were quickly extracted and the weights of spleen and thymus were measured. The small intestine was fixed with 10% neutral formalin buffer. After wrapping with paraffin, the pathologic slice of the small intestine was prepared.

2) Measurement of Cytokines and Immunoglobulins in Serum

The amount of immunoglobulins, IgG, IgG1, IgG2a, IgA and IgE in serum specific to ovaalbumin were measured by ELISA method by using a 96-well coated plate and various anti-globulin antibodies.

3) Immunohistological Evaluation on Small Intestine

After wrapping a slice of small intestine with paraffin by the conventional method, the pathologic slice (5 μm thick) of the small intestine was prepared. Then after removal of paraffin, the immue stain was carried out by using CD4, CD8, INF-γ, and IgA antibody. The numbers of CD4 and CD8 positive cells, INF-γ and IgA secreting cells were counted.

2. Result

1) Effect of β-glucan to Body Weight, the Amount of the Consumed Feed and Systemic Reaction when Ovaalbumin Immunization Immune Reaction

On the β-glucan taken mouse group and the control mouse group, in regard to the change of body weight after 2 weeks, the change of body weight for 1 week after taking ovaalbumin and the change of body weight by immune induction of immunization, the differences between the change of body weight after administration of ovaalbumin (primary immunization) and the change of body weight after administration of ovaalbumin (secondary immunization) were not observed comparing with the no immunized group (the normal mouse group).

In the secondary immunizations by ovaalbumin at 1st and 2nd, it was observed that the amount of the consumed feed drastically decreased in the β-glucan administration group comparing with the normal mouse group (the mouse group not immunized). Namely, evanescent allergic syndrome was observed in both. In 1st immunization of ovaalbumin, the feed consumption decreased in the β-glucan administration group comparing with the normal group. In 2nd immunization, the feed consumption decreased in the control group, allergic syndrome was observed as in 1st immunization, but in the β-glucan administration group (feeds containing 1.0% and 0.5%) the feed consumption increased comparing with the control group and it was almost the same as in the normal mouse group. Namely the allergic syndrome was drastically alleviated.

In regard to systemic reactivity accompanying with 1st and 2nd immunization, by this immunization (2nd immunization), the action of shakes, pillo-election, and crouching of the mice were markedly observed. These actions were not cured even 12 hours later and were not cured by β-glucan administration. By this immunization (2nd immunization), the actions of shakes pillo-election, and crouching of the mice were recovered by taking β-glucan (0.5% and 1.0% contained feed) 4 hours later after administration of ovaalbumin.

2) Effect of β-glucan to the Weights of Spleen and Thymus when Immune Reaction of Ovaalbumin Immunization

The weight of spleen increased by ovaalbumin immunization comparing with the normal mouse group (the no ovaalbumin administration group). On the other hand, the weight of thymus decreased. In the β-glucan administration group, comparing with the control mouse group, in β-glucan 1.0% feed consumption group, the weight of spleen was further increased and the weight of thymus decreased. From this fact, it was considered that immuno function from spleen was promoted by administration of β-glucan (Table 11).

TABLE 11
The change of spleen and thymus
in ovaalbumin immunized mouse

| Treated mice | Number | Spleen (mg) | Thymus (mg) |
|---|---|---|---|
| Normal mice | 7 | 90.2 ± 1.5* | 47.2 ± 2.2* |
| Control (0%) | 8 | 113.9 ± 2.9# | 35.4 ± 1.7# |
| β-glucan 0.25% | 7 | 112.7 ± 2.9# | 33.7 ± 2.8# |
| β-glucan 0.5% | 7 | 119.4 ± 5.8# | 30.6 ± 1.5# |
| β-glucan 1.0% | 7 | 127.0 ± 5.2#* | 28.7 ± 1.2#* |

*For Control mouse (P < 0.05),
#Normal mouse (P < 0.05)
The significance assay was conducted by the multiple assay of Fisher's
protected LSD.

3) Effect of β-glucan to Production of Ovaalbumin Reactive Immunoglobulin in Blood by Ovaalbumin Immunization (Secondary Antibody)

It became clear that antibody-productions by ovaalbumin immunization (IgG, IgG1 (immunoglobulin derived from Th1 cells), IgG2a (immunoglobulin from Th2 cells), IgA and IgE) significantly increased in the control mouse administration group, comparing with the normal mouse group, and by oral administration of ovaalbumin, secondary antibody production was induced and immune reaction was induced. Furthermore, the production of IgG, IgG1, IgG2a and IgA were not effected by taking β-glucan comparing with the control mouse group (Table 3).

On the other hand, IgE production related to allergic reaction was observed to decrease in the β-glucan administration group (1.0% mixed feed) (Table 12). The fact suggested the possibility which β-glucan had antiallergic activity.

TABLE 12
Effect of β-glucan to antibody specific to ovaalbumin
in blood of ovaalbumin immunized mouse

| | | Ovaalbumin specific antibody in blood of | | | | |
|---|---|---|---|---|---|---|
| Treated | | ovaalbumin immunized mouse (OD × 100) ¹⁾ | | | | |
| mice | No. | IgG | IgG1 | IgG2a | IgA | IgE |
| Normal | 7 | 17.0 ± 1.9# | 0.6 ± 0.1# | 0.3 ± 0.1# | 13.7 ± 3.2# | 0.9 ± 0.2 |
| mice | | | | | | |
| Control | 8 | 47.2 ± 8.6# | 43.8 ± 11.3# | 7.7 ± 3.8# | 23.5 ± 2.3# | 1.4 ± 0.3 |
| (0%) | | | | | | |
| β-glucan | 7 | 48.1 ± 8.7# | 45.8 ± 13.5# | 7.9 ± 3.5# | 25.6 ± 3.5# | 0.8 ± 0.2 |
| 0.25% | | | | | | |
| β-glucan | 7 | 45.2 ± 9.8# | 40.6 ± 13.1# | 4.0 ± 1.2# | 20.8 ± 4.1# | 1.0 ± 0.2 |
| 0.5% | | | | | | |
| β-glucan | 7 | 47.1 ± 7.8# | 34.0 ± 10.7# | 7.1 ± 3.0# | 24.7 ± 2.9# | 0.3 ± 0.1#* |
| 1.0% | | | | | | |

¹⁾ Values on each antibody show absorbance
*For Control mouse (P < 0.05),
#Normal mouse (P < 0.05)
The significance assay was conducted by the multiple assay of Fisher's protected LSD.

4) Effect of β-glucan to Small Intestine Immunoreaction by Ovaalbumin Immunization (Immunohistological Evaluation)

As shown in Table 13, by taking ovaalbumin, the numbers of lymphocyte CD4 positive cells and CD8 positive cells in small intestine significantly increased in the β-glucan consumption group (0.5% and 1.0% mixed food) in comparing with the control mouse group. This fact showed the possibility that β-glucan further potenciated the immune inflammatory in small intestine due to ovaalbumin immunization and that it reacted as bioprotective reaction to the foreign matter. Namely there are possibilities that Th1 cells of CD4 are activated to produce IFN-γ, etc. and it works on B cells to produce immunoglobulin and exclude pathogenic microorganisms. Furthermore, it is possible that the increase of the number of CD8 positive cells controls excess immunoinflammatory reaction in intestinal tube and exclusion of introduction of excess antibody.

Furthermore, it was suggested that CD4 positive cells were observed in Th1 cells and Th2 cells, and activated helper T cells. Though the number of IgA secreting cells was not significantly increased in the β-glucan administration group, comparing with the control mouse group, the tendency of the promotion was found. On the other hand, CD8 positive cells showed the activation of killer cells induced due to the activation of Th1 cells and it was suggested that the increase of the number of CD8 positive cells significantly activated Th1 cells system comparing with Th2 cells system. From this fact, it was suggested that β-glucan controlled allergic reaction.

TABLE 13
Effect of β-glucan to the numbers of IgA secreting cells and
the numbers of lymphocyte CD4 positive cells and CD8 positive cells
and in the small intestine of ovaalbumin immunized mouse

| | | Number of small intestine of | | |
|---|---|---|---|---|
| | | ovaalbumin immunized mouse | | |
| | | (Cell number per field) | | |
| Treated mice | Number | CD4 | CD8 | IgA |
| Normal mice | 7 | 54 ± 4 | 229 ± 21 | 40 ± 5 |
| Control (0%) | 8 | 44 ± 7 | 204 ± 25 | 32 ± 4 |
| β-glucan 0.25% | 7 | 77 ± 9* | 249 ± 37 | 30 ± 4 |
| β-glucan 0.5% | 7 | 100 ± 5*# | 386 ± 17*# | 39 ± 4 |
| β-glucan 1.0% | 7 | 133 ± 12*# | 402 ± 22*# | 41 ± 4 |

*For Control mouse (P<0.05),
#Normal mouse (P < 0.05)
The significance assay was conducted by the multiple assay of Fisher's protected LSD.

### CONCLUSION

From the above results, it is suggested that β-glucan exhibits the function to control inflammatory with immunoreaction to food antibody, ovaalbumin and to maintain the biodefensive reaction, when β-glucan is taken.

1. An aqueous β-1,3-1,6-D-glucan solution, wherein ¹HNMR spectra on said solution containing 1N aqueous sodium hydroxide heavy solution have two signals of 4.7 ppm and 4.5 ppm and the viscosity of said solution at 30° C., pH5.0, and its concentration 0.5% (w/v) is 50 cP ([mPa.s]) or less.

2. A process for preparing an aqueous β-1,3-1,6-D-glucan solution which comprises adding an alkali or its aqueous solution to microorganism-culture medium containing β-1,3-1,6-D-glucan of claim 1, adjusting pH to 12 or more than 12 to make the viscosity of the medium lower and then, separating and removing the insoluble materials including a microorganism, and further removing metal ions, so that the metal ion concentration becomes 120 mg/100 ml or less.

3. The process for preparing the aqueous β-1,3-1,6-D-glucan solution according to claim 2, wherein the microorganism-culture medium is one of Aureobasidium pullulans GM-NH-1A1 (deposited number: FERM BP-10294) or Aureobasidium pullulans GM-NH-1A2 (deposited number: FERM BP-10295).

4. A process for preparing β-1,3-1,6-D-glucan in powder with high purity which comprises dialyzing and concentrating the aqueous β-1,3-1,6-D-glucan solution of claim 1, adding an alcohol to its concentrate to precipitate β-1,3-1,6-D-glucan, separating thus obtained a slurry consisting of β-1,3-1,6-D-glucan/alcohol/water by a separating funnel and spray-drying the precipitated slurry-D-glucan.

5. A process for preparing β-1,3-1,6-D-glucan in powder with high purity which comprises dialyzing the aqueous β-1,3-1,6-D-glucan solution of claim 1, concentrating it under reduced pressure and spray-drying the residue.

6. An pharmaceutical composition comprising the aqueous β-1,3-1,6-D-glucan solution of claim 1 or the β-1,3-1,6-D-glucan prepared by drying it.

7. A healthy food or a health promoting food comprising the aqueous β-1,3-1,6-D-glucan solution of claim 1 or the β-1,3-1,6-D-glucan prepared by drying it.

8. A cosmetic comprising the aqueous β-1,3-1,6-D-glucan solution or β-1,3-1,6-D-glucan of claim 1 or β-1,3-1,6-D-glucan prepared by drying it.

9. A method for treating malignancy neoplasm, allergic disease or a disease caused by INF-γ inducing which comprises administering to a patient the aqueous β-1,3-1,6-D-glucan solution or β-1,3-1,6-D-glucan of claim 1 or the β-1,3-1,6-D-glucan prepared by drying it in an effective amount.

10. A method for inhibiting malignancy neoplasm metastasis, inducing INF-γ or activating macrophage which comprises administering to a patient the aqueous β-1,3-1,6-D-glucan solution or β-1,3-1,6-D-glucan of claim 1 or the β-1,3-1,6-D-glucan prepared by drying it in effective amount.
